(19) United States
(12) Patent Application Publication
(10) Pub. No.: US 2022/0010076 A1
(43) Pub. Date: Jan. 13, 2022

(54) GREEN METHOD TO PREPARE PLAIN WATER-BASED POLYSACCHARIDE CHITOSAN SOLUTIONS

(71) Applicant: VIRGINIA COMMONWEALTH UNIVERSITY, RICHMOND, VA (US)

(72) Inventors: Boxuan LI, Richmond, VA (US); Juan WANG, Richmond, VA (US); Hu YANG, Glen Allen, VA (US)

(73) Assignee: VIRGINIA COMMONWEALTH UNIVERSITY, RICHMOND, VA (US)

(21) Appl. No.: 17/297,853

(22) PCT Filed: Nov. 27, 2019
(86) PCT No.: PCT/IB2019/060256

§ 371 Date: May 27, 2021

Related U.S. Application Data
(60) Provisional application No. 62/772,031, filed on Nov. 27, 2018.

(51) Int. Cl. C08J 3/05; A61K 47/36; C08L 5/08; A61K 8/73; A23L 29/275

(52) U.S. Cl. CPC C08J 3/05; A61K 47/36; C08L 5/08; A23V 2002/00; A23L 29/275; C08J 2305/08; A61K 8/736

(57) ABSTRACT

A method is provided for producing a composition comprising chitosan and water, comprising dissolving chitosan powder in an ionic liquid, to prepare a first composition comprising chitosan and ionic liquid; and contacting the first composition with water under conditions sufficient to solvent-exchange all or substantially all of the ionic liquid with water; to form a composition comprising chitosan and water. Compositions produced thereby and methods of using the compositions are provided.

### RELATED APPLICATIONS

This application claims the benefit of U.S. Application 62/772,031, filed Nov. 27, 2018, the entire contents of which are hereby incorporated by reference.

### FIELD OF THE INVENTION

The invention relates to water-based chitosan compositions, methods for their preparation, and methods for using them.

### BACKGROUND

Chitosan is an important and abundant natural-based polymer, and it has broad applications. However, dissolving chitosan into plain water is a serious challenge, which limits biomedical and other applications of chitosan materials. Chitosan, the deacetylated derivative of chitin extracted from the exoskeletons of crabs and shrimps, is one of the most important natural-based polymers. It has been broadly included in cosmetic and food products as a common constituent, in pharmaceutical formulations as an excipient, or in biomedicines as a carrier by its low toxicity, biocompatibility and biodegradability. Nonetheless, chitosan exhibits low solubility in water and most organic solvents because of strong intermolecular and intramolecular hydrogen-bonding interactions among chitosan macromolecular chains. The solvent systems currently available for dissolving chitosan are few and have distinct limitations. Traditionally, aqueous acid, alkali, or alkali-urea systems are commonly used, but they can be corrosive or toxic, or otherwise have limitations for biomedical and other applications. Even a trace amount of acid or alkali residues may exert biological influences negatively, posing a challenge to purification and raising safety concerns.

The pioneering study of dissolving natural polysaccharide in ionic liquids (ILs) in 2002 by Swatloski (J. Am. Chem. Soc. 2002, 124, 4974-4975. DOI: 10.1021/ja025790m) has stirred up much interest for dissolution of chitosan. As environmentally benign solvents with the advantages of being almost non-volatile, high thermal and chemical stability, and easy to recycle, ionic liquids can be suitable alternatives for dissolving chitosan through breaking and disrupting the hydrogen-bonded networks within chitosan molecules (Silva, S. S., et al., Green Chem. 2017, 19, 1208-1220. DOI: 10.1039/c6gc02827f; Lu, L., et al., Adv. Mater 2010, 22, 3745-3748. DOI: 10.1002/adma.201001134). Usually, the dissolution of chitosan by ILs needs high temperatures (over 100° C. for ˜10 wt %) (Sun, X., et al. RSC Adv. 2014, 4, 30282-30291. DOI: 10.1039/c4ra02594f). From a biological perspective, the toxicity of ILs has yet to be fully elucidated, thus requiring careful safety assessment for the biomedical applications of chitosan-ILs systems. Although water-soluble chitosan can be obtained through chemical modification or degradation, the processes are usually tedious, and impurities are introduced (Roberts, G. A. F., In Chitin Chemistry; Mcmillan Press Ltd.: London, 1992).

Dissolving chitosan into plain water is a longed-for solution to eliminate the solvent effects in chitosan bio-based and other applications, but heretofore it has not been possible.

### BRIEF DESCRIPTION OF THE FIGURES

FIG. 1. A schematic showing an exemplary embodiment of converting chitosan from a solid powder to a plain water-based pseudo-solution using an eco-friendly method.

FIG. 2 (a)-(d). Experimental results picturing various exemplary embodiments. (a) The HMW chitosan/EMIM Ac system undergoes a significant swelling during the process of solvent exchanging with water for 7 days. (b) Chitin/EMIM Ac and cellulose/EMIM Ac systems undergo a limited swelling through the solvent exchanging with water. (c) The obtained plain water-based chitosan solution shows the Tyndall effect. (d) LMW and MMW chitosan have been successfully processed to form plain water-based solutions by using this method.

FIG. 3 (a)-(c). EMIM Ac has been successfully removed as confirmed by HPLC. Compared with 10 mM EMIM Ac (a) and 1 mM EMIM Ac (b), EMIM Ac became undetectable following extensive solvent exchange with water (c).

FIG. 4 (a)-(d). (a) Hydrodynamic size distribution and (b) zeta potential of an exemplary chitosan pseudo solution. Inset: Hydrodynamic size distribution of chitosan in acid (pH=2.2; Prepared concentration: 2.4 mg/mL; Peak: ˜6.1 nm). (c) Hydrodynamic size distribution of an exemplary chitosan pseudo solution diluted to 1.2 mg/mL by PBS (black, right-hand peak) and 0.85% NaCl aqueous solution (red, left-hand peak) at 37° C. for 24 h; and (d) zeta potential of an exemplary chitosan pseudo solution diluted to 1.2 mg/mL by PBS (black, left-hand bar) and 0.85% NaCl aqueous solution (red, right-hand bar) at 37° C. for 24 h.

FIG. 5 (a)-(b). (a) Dependence of the viscosity on the shear rate. Inset: Injection of an exemplary chitosan pseudo solution doped Rhodamine B through a 0.26 mm inner diameter needle. (b) Oscillatory frequency sweep. Symbols: •, storage modulus (G′); ∘, loss modulus (G″).

FIG. 6. TEM image of exemplary chitosan nanoparticles after an exemplary chitosan pseudo-solution was air-dried.

FIG. 7 (a)-(b). SEM micrographs of the porous structures of an exemplary chitosan foam after freeze-drying at different magnifications (a, b). Inset of (a): image of the chitosan foam. Inset of (b): Nano/submicron fibrous filaments, scale bar: 500 nm.

FIG. 8. Swelling behavior of an exemplary chitosan foam after freeze-drying of an exemplary chitosan pseudo-solution in PBS at 37° C. for 30 days.

FIG. 9 (a)-(d). One proposed possible mechanism for preparing a plain water-based chitosan solution. Chitosan molecular chains form a hydrogen-bonded network, making solubilizing chitosan a challenge (a). Ionic liquid EMIM Ac solubilizes chitosan by breaking the hydrogen-bonded network (b). The chitosan-EMIM Ac system undergoes freezing and solvent exchange with water (c), leading to the removal of IL molecules and the protonation of chitosan's amine groups, which eventually form a plain water-based chitosan pseudo-solution (d).

FIG. 10. A schematic showing one embodiment of an exemplary process for preparing the water-based chitosan solution.

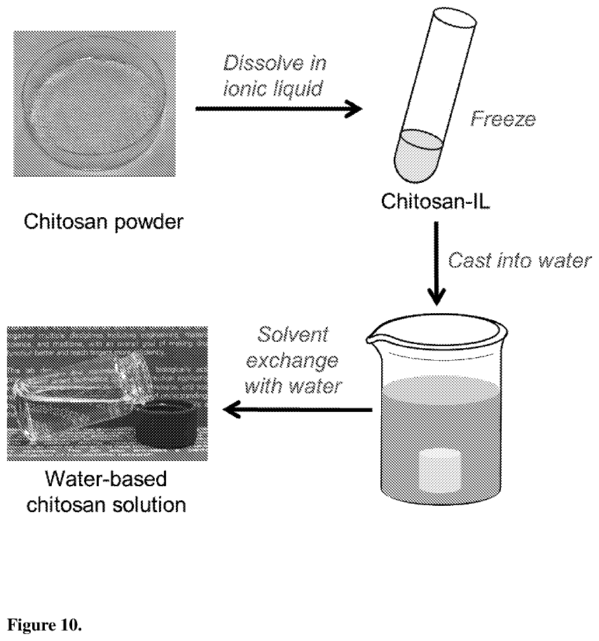

FIG. 11. Shows pictorially one embodiment of an exemplary chitosan film prepared by evaporating water of the chitosan pseudo solution.

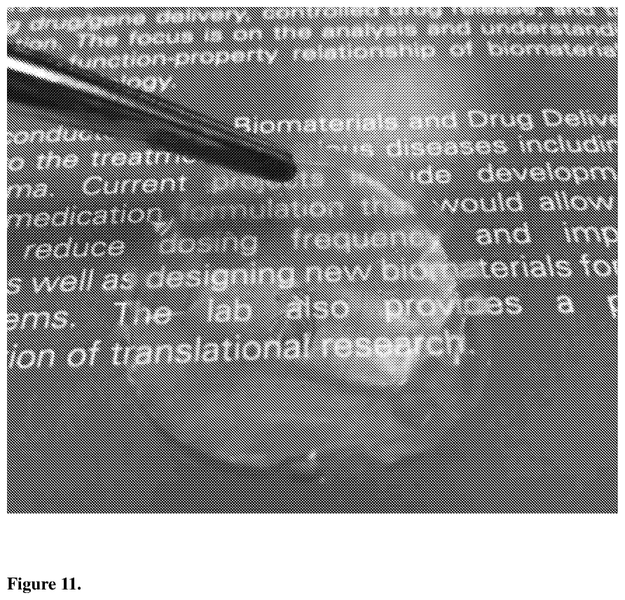

FIG. 12. Shows pictorially one embodiment of an exemplary chitosan foam prepared by freeze-drying of the chitosan pseudo solution.

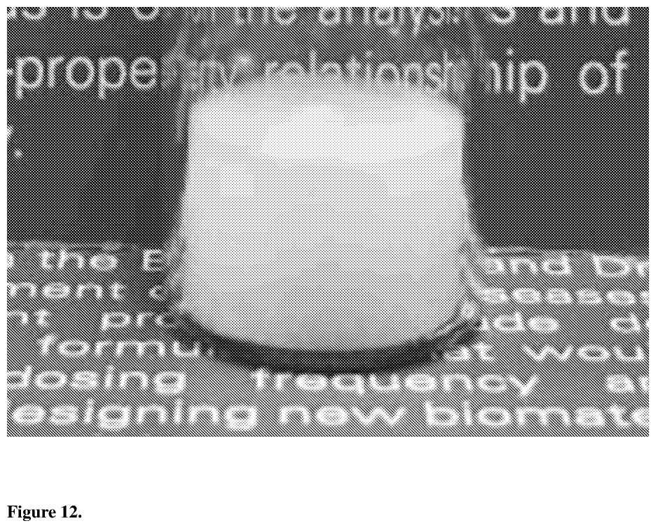

### BRIEF DESCRIPTION OF THE SEVERAL EMBODIMENTS

In one aspect, a simple eco-friendly method to prepare plain water-based chitosan pseudo-solutions and chitosan compositions is provided.

In another aspect, an eco-friendly dissolution method to obtain a plain water-based chitosan solution is provided, which includes dissolving chitosan into ionic liquid to form a chitosan/ionic liquid mixture, freezing the chitosan/ionic liquid mixture to form a frozen chitosan/ionic liquid mixture, solvent exchanging with plain water at room temperature with the frozen chitosan/ionic liquid mixture, to form a uniform and stable chitosan solution in water with nano-sized chitosan solutes, namely water-based chitosan pseudo-solution.

In another aspect, a method is provided for producing a composition comprising chitosan and water, comprising:

dissolving chitosan powder in an ionic liquid, to prepare a first composition comprising chitosan and ionic liquid; and

contacting the first composition with water under conditions sufficient to solvent-exchange all or substantially all of the ionic liquid with water;

to form a composition comprising chitosan and water.

In another aspect, a composition is provided, comprising chitosan and water, prepared by a process comprising:

dissolving chitosan powder in an ionic liquid, to prepare a first composition comprising chitosan and ionic liquid; and

contacting the first composition with water under conditions sufficient to solvent-exchange all or substantially all of the ionic liquid with water;

to form a composition comprising chitosan and water.

In another aspect, a composition is provided, comprising nano-sized chitosan solutes and water.

The overall process is eco-friendly. The new method surprisingly and unexpectedly augments the quality and processability of chitosan solutions used in manufacturing and bioprocessing and promotes the biomedical and other applications of chitosan-based products.

### Detailed Description of the Several Embodiments

In one embodiment, chitosan is first dissolved into an ionic liquid or mixture of one or more ionic liquids, then frozen. Afterward, solvent exchange is carried out with plain water at room temperature, hence obtaining a stable dispersion of nano-sized chitosan in plain water. In this process, it is believed that the hydrogen-bonded network of chitosan is disrupted by the ionic liquid to free the amines that participate in hydrogen bonding. The intermediate freezing step is believed to prevent the dissolved chitosan from reconnecting with hydrogen-bonding interactions and undesirably aggregating. The solvent exchange step is believed to lead to the protonation of chitosan's amine groups, which, in turn, promotes chitosan solvation. After removing all or substantially all of the ionic liquid, the vortexing step is believed to help form a stable and uniform nano-sized chitosan solution. This method unexpectedly and surprisingly provides uniform and stable plain water-based chitosan solutions. The solutions herein desirably open up opportunities to use plain-water based chitosan solution for applications in the fields of pharmaceutical and biomedicine and others.

The chitosan and chitosan powder are not particularly limited. The chitosan may be low, medium, or high molecular weight, or a combination thereof. It may be modified in accordance with known methods, or it may be unmodified. In one embodiment, the chitosan is unmodified. One or more than one type of chitosan may be used.

The molecular weight (wt. avg.) of the chitosan may suitably range from about 5,000 to about 400,000 Da. This range includes all values and subranges therebetween, including 5, 10, 15, 20, 25, 30, 35, 40, 45, 50, 55, 60, 75, 80, 85, 90, 95, 100, 110, 150, 190, 200, 250, 300, 310, 325, 350, 375, 390, and 400 kDa, or any combination thereof. In one embodiment, the chitosan is low molecular weight (50,000-190,000 Da or lower). In another embodiment, the chitosan is medium molecular weight (190,000-310,000 Da). In another embodiment, the chitosan is high molecular weight (310,000-375,000 Da or higher).

The ionic liquid is not particularly limited, and any ionic liquid capable of dissolving chitosan or chitosan derivative may be used. In one embodiment, the ionic liquid is a hydrophilic ionic liquid or combination of two or more hydrophilic ionic liquids. Non-limiting examples of ionic liquid (IL) include one or more of 1-allyl-3-methyl-imidazolium bromide ([Amim][Br]), 1-allyl-3-methyl-imidazolium chloride ([Amim][Cl]), 1-butyl-3-methyl-imidazolium acetate ([Bmim][Ac]), 1-butyl-3-methyl-imidazolium chloride ([Bmim][Cl]), 1-ethyl-3-methyl-imidazolium acetate ([Emim][Ac]), 1-ethyl-3-methyl-imidazolium chloride ([Emim][Cl]), 1-ethyl-3-methyl-imidazolium dimethyl phosphate ([Emim][Me₂PO₄]), 1-carboxymethyl-3-methylimidazolium chloride ([ImimCOOH][Cl]), hydrophilic ionic liquid; hydrophilic ionic liquid system; or any combination thereof.

In one embodiment, the ionic liquid is one or more of 1-allyl-3-methyl-imidazolium bromide; 1-allyl-3-methyl-imidazolium chloride; 1-butyl-3-methyl-imidazolium chloride; 1-ethyl-3-methyl-imidazolium chloride; or any combination thereof.

In one embodiment, the ionic liquid is one or more of 1-butyl-3-methyl-imidazolium acetate; 1-ethyl-3-methyl-imidazolium acetate; or any combination thereof.

In one embodiment, the ionic liquid is 1-ethyl-3-methyl-imidazolium acetate, either alone or in combination with another ionic liquid. In one embodiment, the ionic liquid is 1-ethyl-3-methyl-imidazolium acetate.

In one embodiment, the ionic liquid may be recycled and reused.

The content of chitosan in the chitosan-IL is not particularly limited, and may suitably range from 0.001 to about 50 wt. %. This range includes all values and subranges therebetween, including 0.001, 0.002, 0.005, 0.007, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50 wt. % or any combination thereof. In one embodiment, concentrations of about 10 wt. % are used.

The temperature at which the chitosan is dissolved in the ionic liquid is not particularly limited, but temperatures of room temperature or higher are generally used. Non-limiting dissolution temperatures of 25, 30, 40, 50, 60, 70, 80. 90. 100, 110, 120, 130° C. or higher may be used. In one embodiment, dissolution temperatures of about 120° C. are used.

The freezing temperature is not particularly limited, so long as it is suitable to freeze or solidify the chitosan-ionic liquid mixture. For example, temperatures of 0° C. or lower may be suitably employed. In one embodiment, a temperature of 0 to −20° C. is employed to freeze the mixture. In one embodiment, and temperature of −20° C. is used.

The time of freezing, i.e., time of storage at freezing temperature, is not particularly limited and may range from a few hours or less to 24 hours or more. This range includes all values and subranges therebetween, including 1, 2, 3, 4, 5, 6, 7, 8, 9, 10, 11, 12, 13, 14, 15, 16, 17, 18, 19, 20, 21, 22, 23, and 24 hours. Generally, overnight storage at freezing temperature is suitable, but other times may also be suitable.

After freezing, the frozen chitosan/IL is then contacted with an excess amount of water, e.g., deionized water, to effect solvent exchange. Generally, the solvent-exchange water is at room temperature, but other temperatures may be used. For example, the temperature of the solvent-exchange may range from 1 to 100° C. This range includes all values and subranges therebetween, including 1, 5, 10, 15, 20, 25, 30, 35, 40, 45, 50, 55, 60, 65, 70, 75, 80, 85, 90, 95 and 100° C. or any combination thereof.

The water-contacting for the solvent exchange step may be carried out by submersion, dialysis, stirring, shaking, vortexing, flowing water, stable water, water vapor, or a combination thereof. In one embodiment, the contacting is via submersion of the frozen chitosan/IL in non-flowing water at room temperature. In one embodiment, the frozen chitosan/IL was submerged in a beaker containing plain deionized water for solvent exchange at room temperature.

The solvent-exchange water may be refreshed during the process. Further, the unabsorbed water may be decanted and replaced until all or substantially all of the ionic liquid is removed, as shown, for example, by HPLC. That is, the solvent exchange is desirably carried out until no ionic liquid is detected in the solvent-exchange water. In one embodiment, the solvent exchange is complete, and all of the ionic liquid in the chitosan/IL mixture is replaced by water.

The time of solvent-exchange is not particularly limited, so long as it is sufficient to remove all or substantially all of the ionic liquid from the chitosan/IL mixture and replace it with water. In one embodiment, the solvent exchange is carried out for a period of a few days or less to a week, e.g., until no further ionic liquid is detected. This range includes all values and subranges therebetween, including 0.1, 0.5, 1, 2, 3, 4, 5, 6, and 7 days or longer.

After solvent exchange and decanting of unabsorbed water, the remaining swollen chitosan gel-like solution may be subjected to vortexing, to obtain pseudo (colloidal) solution. The time and frequency of vortex and dispersing are not particularly limited. As an example, the vortexing may be carried out for a few minutes or more at an rpm of 3,000-40,000 rpm. In one embodiment, the vortexing is carried out for about three minutes at 30,000 rpm.

The size of the chitosan solutes in the obtained pseudo colloidal solution suitably ranges in the nanosize, from 1 to 1000 nm. This range includes all values and subranges therebetween, including 1, 2, 3, 4, 5, 6, 7, 8, 9, 10, 20, 30, 40, 50, 60, 70, 80, 90, 100, 200, 300, 400, 500, 600, 700, 800, 900 and 1000 nm. In one embodiment, the size ranges from about 1-500 nm, 5-200 nm, 10-100 nm, or combination thereof.

The concentration of the chitosan in the pseudo solution is not particularly limited. For example, the concentration of chitosan may range from 0.000001 to 20 mg/ml and higher. This range includes all values and subranges therebetween, including 0.000001, 0.000002, 0.000003, 0.000004, 0.000005, 0.000006, 0.000007, 0.000008, 0.000009, 0.00001, 0.00002, 0.00003, 0.00004, 0.00005, 0.00006, 0.00007, 0.00008, 0.00009, 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.4, 1.6, 1.8, 2, 2.2, 2.4, 2.6, 2.8, 3, 4, 5, 6, 7, 8, 9, 10, 12, 14, 16, 18 and 20 mg/ml. Other concentrations are possible so long as the chitosan solutes remain in the 1-1000 nm size range.

The obtained pseudo colloidal solution is desirably stable in that the chitosan solutes maintain or substantially maintain their size and do not aggregate.

The pseudo colloidal solution may be used as prepared, or it may be further processed. For example, in one embodiment, a transparent and flexible chitosan film can be obtained from the chitosan pseudo solution by evaporating water. In another embodiment, a foam material of chitosan can be obtained by freeze-drying the chitosan pseudo solution. The porous microstructures of the chitosan foam have been studied by SEM, and the inventors have found that chitosan nanofibers with various morphologies permeate through the whole porous chitosan foam. After removal of water, the hydrogen-bonded networks within chitosan molecules can recover, and the freeze-dried chitosan will not be redissolved into water environment and are stable for at least 30 days.

The chitosan solution has a good processability to be injected, cast, 3D printed and so on for a liquid, injectable gel, gel, film, foam, 3D structure, and the like. The chitosan solution has great potential biomedical and pharmaceutical applications. For example, because the ionic liquids can be designed and they can dissolve many pharmaceuticals, using this method both hydrophilic and hydrophobic drugs could be encapsulated into chitosan for drug delivery, cell adhesion, and tissue engineering, to name a few. The chitosan solution can also be applied in the fields of water treatment, food packaging and cosmetic products. Moreover, this method is also applicable to a wide range of polysaccharide-based materials or polyion (polycation/polyanion) materials, such as chitin-based materials, cellulose-based materials and so on.

In one embodiment, chitosan in powder form is first dissolved in an ionic liquid (IL). The formed chitosan/IL solution is frozen. The frozen chitosan/IL solution is then contacted with, e.g., submersed in or otherwise exposed to, an excess amount of plain water equilibrated at room temperature to initiate the solvent exchange process (25° C.). Throughout the solvent exchange process, the chitosan/IL system continuously swells and expands in volume and eventually becomes fluidic. The process is not terminated until the ionic liquid becomes undetectable in the water phase, for example, by HPLC. Upon the removal of unabsorbed water phase and vortexing, a clear chitosan pseudo-solution forms. As a result of extensive solvent exchange with water, the final chitosan concentration in water is reduced many-fold as compared to its concentration in the ionic liquid.

In one embodiment, the method includes:

dissolving chitosan powder in an ionic liquid, to prepare a first composition comprising chitosan and ionic liquid;

freezing the first composition, to form a frozen composition comprising chitosan and the ionic liquid;

contacting the frozen composition with excess water to solvent-exchange all or substantially all of the ionic liquid with water, to form a composition comprising chitosan and water; and

vortexing the composition comprising chitosan and water;
to form a composition comprising water and chitosan solutes having a size in the range of 1 to 1,000 nm.

In one embodiment, after dissolving the chitosan in the ionic liquid, the resulting mixture is frozen prior to contacting it with water. In one embodiment, the excess water if any is desirably removed from the chitosan and water composition.

In one embodiment, the composition prepared by the process consists essentially of chitosan and water.

In one embodiment, the composition prepared by the process consists of chitosan and water.

In one embodiment, the composition of chitosan and water is in the form of solution, pseudo-solution, colloid, liquid, hydrogel, injectable gel, film, foam, nanofiber, porous scaffold, or particle.

In one embodiment, the composition comprising chitosan and water is optically transparent.

The composition of chitosan and water may be used alone, or it may include one or more benefit compounds. The benefit compound is not particularly limited. Non-limiting examples of benefit compounds include one or more of pharmaceutically active ingredient, drug, hydrophobic drug, hydrophilic drug, biomolecule, medicine, cosmetic, food, or combination thereof. For example, the pharmaceutically active ingredient may be a compound for treating infection, glaucoma, or other malady or for cell adhesion, tissue engineering and the like.

If desired, a benefit compound can be dissolved into ionic liquid along with chitosan or into plain-water-based chitosan solution after the ionic liquid has been removed. The loading of benefit compound can be facilitated with heating or at low temperature with vortexing depending on the property of benefit compounds.

In one embodiment, the chitosan and water composition includes nano-sized chitosan solutes and water.

In one embodiment, according to dynamic light scattering measurements, the chitosan water solution contains uniform nano-sized chitosan in the so-called pseudo (colloidal) solution. In one embodiment, it exhibits a typical Tyndall effect and is relatively pH-neutral. This is advantageously different from systems in which chitosan is dissolved in an acidic buffer solution to form a pseudo solution, which are extremely acidic and require neutralization prior to their use in formulations. The plain water-based chitosan pseudo-solutions described herein show good colloidal stability. In one embodiment, over a period of five days, they do not form particle aggregation and maintain a relatively unchanged zeta potential. In one embodiment, the solution has a shear thinning property in that the viscosity of the chitosan pseudo-solution decreases dramatically by four orders of magnitude over the shear rate increase from 10⁻² to 10³ l/s. In an embodiment, the water-based chitosan pseudo-solution is injectable. It can be pushed out a syringe without clogging the needle. In one embodiment, the oscillatory frequency sweep test elucidates that the solution has a slightly higher storage modulus (G′) than loss modulus (G″), and G′ is practically independent of frequency, the features typically displayed in a weak hydrogel. The gel properties of chitosan pseudo-solution are believed to be attributed to the significantly weakened hydrogen-bonding interactions among nano-sized chitosan particles. The present inventors have successfully processed chitosan of different molecular weights to form water-based pseudo-solutions, suggesting the robustness of the approach. Upon the removal of water from the pseudo-solution via the freeze drying process, the chitosan particles reconnect to form a porous foam material. The present inventors also observed that nano/submicron fibrous filaments randomly permeate through the foam, further indicating the success of dispersing chitosan to nanostructures. Conversely, the recovered chitosan becomes insoluble in water again in large part due to the restored hydrogen-bonding networks among the chitosan molecules. In one embodiment, the material remains stable for at least 30 days.

In one embodiment, chitosan is first dissolved into the ionic liquid EMIM Ac and then kept the chitosan/ionic liquid mixture at −20° C. overnight. Afterward, extensive solvent exchange was carried out with plain water at room temperature, hence obtaining a stable dispersion of nano-sized chitosan in plain water. In this process, the hydrogen-bonded network of chitosan is disrupted by the ionic liquid to free the amines that participate in hydrogen bonding. The intermediate freezing step prevents the dissolved chitosan from reconnecting with hydrogen-bonding interactions and undesirably aggregating. The solvent exchange step leads to the protonation of chitosan's amine groups, which, in turn, promotes chitosan solvation. This method opens up opportunities to use plain-water based chitosan solution for applications in the fields of pharmaceutical and biomedicine.

### Examples

Materials and Methods

Materials. Chitosan with high molecular weight (HMW) (310,000˜375,000 Da), medium molecular weight (MMW) (190,000˜310,000 Da), low molecular weight (LMW) (50,000˜190,000 Da), chitin from shrimp shells (practical grade, powder), cellulose (microcrystalline powder) and ionic liquid 1-ethyl-3-methylimidazolium acetate (EMIM Ac, ≥95.0%) were purchased from Sigma-Aldrich. Water was ultrapurified by deionization and filtration before use. Acetonitrile (ACN), trifluoroacetic acid (TFA), sodium phosphate dibasic dihydrate (Na₂HPO₄.2H₂O), citric acid, sodium chloride (NaCl) and phosphate buffered saline (PBS, 10×) were purchased from Thermo Fisher Scientific. Rhodamine B was purchased from Fluka.

Preparation of chitosan pseudo-solutions. Chitosan powders were dissolved in the ionic liquid EMIM Ac and kept at 120° C. to form a transparent and viscous solution at the final concentration of 10 wt %. The obtained chitosan/IL solution was kept at −20° C. overnight. Afterward, the frozen chitosan/IL was submerged in a beaker containing plain deionized water for solvent exchange at room temperature, during which water was refreshed multiple times until EMIM Ac became undetectable by HPLC (mobile phase: ACN/water/TFA 1/1/0.05 (v/v/v). UV detection wavelength: 310 nm.). The unabsorbed water was decanted. The remaining swollen chitosan gel-like solution was subjected to vortexing and dispersing at 30,000 rpm for 3 minutes to form a solution.

For comparison, a chitosan pseudo solution was prepared using the conventional acid method. Briefly, chitosan of appropriate amounts was dissolved in an acid solution (pH=2.2, a mixture of 0.2 M Na₂HPO₄/0.1 M citric acid, 1/26.5 v/v) and stirred overnight at room temperature for complete dissolution. The final concentration was 2.4 mg/mL.

Size and zeta potential measurements. The hydrodynamic size distribution and zeta potential of chitosan pseudo solution were characterized by using a Malvern Zetasizer Nano ZS90 (Malvern Instruments, Malvern, Worcestershire, U.K.).

Rheological property measurements. Rheological experiments were carried out on a Discovery Hybrid Rheometer HR-3 (TA Instruments) using a 20 mm parallel plate geometry at 25° C. An amplitude sweep (not shown) was first performed at a constant angular frequency of 1 rad/s in the strain range of 0.01% to 100%. Within the linear viscoelastic region (LVR), oscillatory frequency sweeps were carried out under 1% strain in the angular frequency range of 0.1 rad/s to 600 rad/s. A viscosity vs. shear rate flow sweep was performed from 0.01 s⁻¹ to 500 s⁻¹.

Transmission electron microscopy (TEM). The TEM images of chitosan nanoparticles were obtained by JEM-1400 Plus TEM at an accelerate voltage of 120 kV. The samples were prepared by drop-coating and air-drying chitosan pseudo-solution on a carbon-coated copper grid.

Scanning electron microscopy (SEM). Freeze-dried chitosan pseudo-solution were immobilized on a stub and sputtering coated by Pt for 90 seconds and then imaged under a scanning electron microscope (Hitachi FE-SEM Su-70, Japan) at the accelerating voltage of 5 kV.

Swelling studies. The stability of the freeze-dried chitosan foam in terms of mass loss in PBS at 37° C. was measured.

pH measurements. The pH measurements were conducted with a pH Benchtop Meter (Fisher, AE 150, U.S.).

Results and Discussion

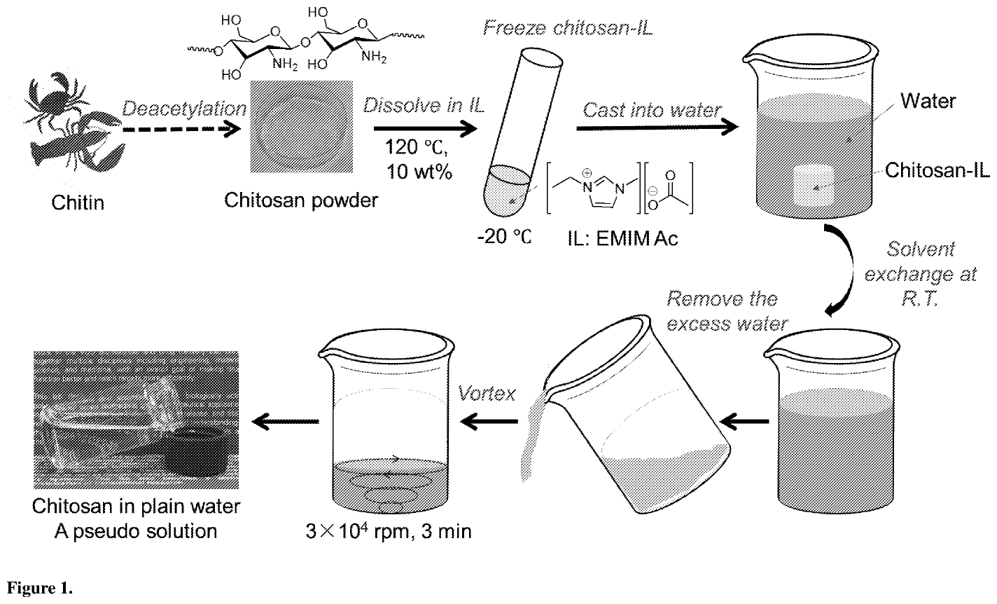

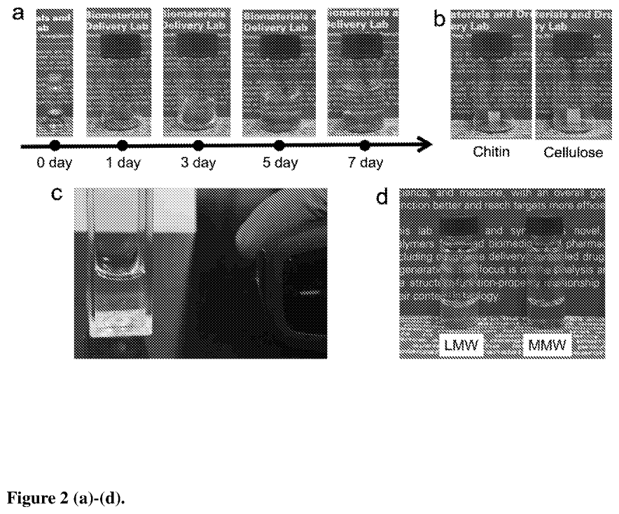

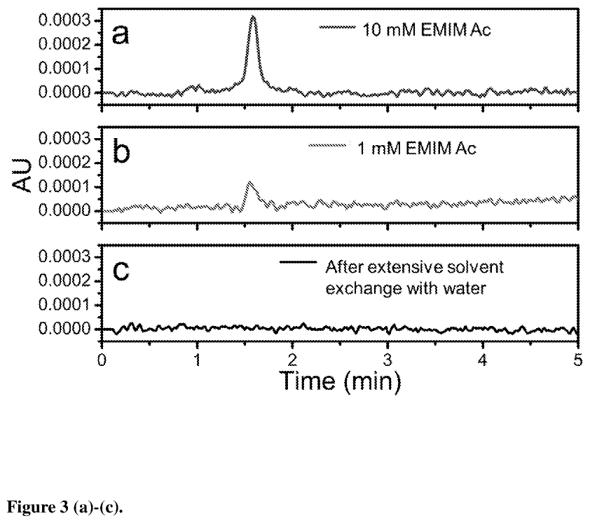

As illustrated in FIG. 1, HMW chitosan in powder form was first dissolved in an ionic liquid (IL) at a high concentration (10 wt %) under 120° C. IL 1-ethyl-3-methylimidazolium acetate (EMIM Ac) was used to dissolve chitosan. The resulting chitosan/IL solution was kept frozen at −20° C. overnight and then submerged in an excess amount of plain water equilibrated at room temperature to initiate the solvent exchange process (25° C.). Throughout the solvent exchange process, it was observed that the chitosan/IL system swelled and continuously expanded in volume (FIG. 2a). It became fluidic at day four. This swelling process was entirely different from the system of chitin/EMIM Ac or cellulose/EMIM Ac, which was characterized by a limited swelling in water (FIG. 2b). Upon the removal of unbound water and subsequent vortexing, a clear water-based chitosan solution formed. It exhibited the Tyndall effect, a common phenomenon for colloidal solutions (FIG. 2c). LMW and MMW chitosan were also processed to form water-based solutions successfully (FIG. 2d), suggesting the robustness of this approach. Ionic liquid EMIM Ac in the solvent exchange process was monitored by using HPLC. At day 7, EMIM Ac became undetectable in the water phase (FIG. 3). Over 70% mass of chitosan was recovered from the final water-based solution.

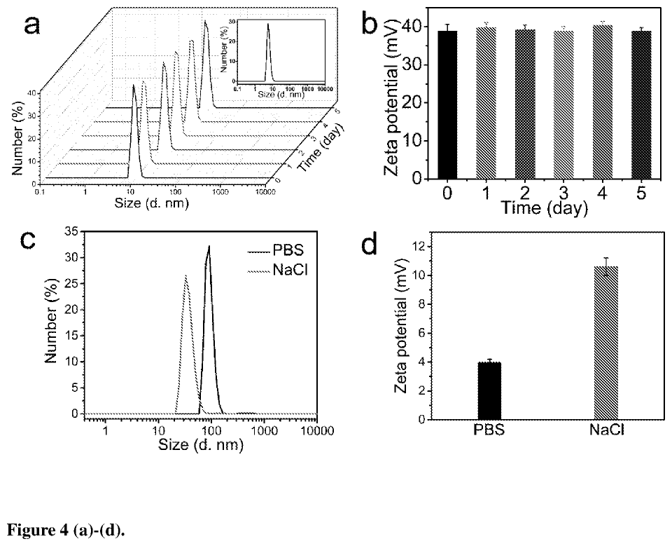

The chitosan water solution contained nano-sized chitosan with a mean diameter of about 9.3 nm (PDI=0.49) (FIG. 4a). In contrast to a true solution containing solutes of a size smaller than 1 nm, a pseudo (colloidal) solution is composed of solutes with the size typically from 1 to 1000 nm. When the solute size is larger than 1000 nm, the solution is considered a suspension. Thus, the formed chitosan solution is the so-called pseudo (colloidal) solution. For a comparison, chitosan dissolved in an acidic buffer solution formed a pseudo-solution (FIG. 4a inset) with nano-sized chitosan and maintained as a water-soluble cationic polyelectrolyte, which was realized through amine protonation at low pH.

The plain water-based chitosan pseudo-solutions showed excellent colloidal stability. Over a period of five days, they did not induce particle aggregation and kept a relatively unchanged high zeta potential about +40 mV (FIG. 4a, b). The stability of the chitosan pseudo solution diluted to 1.2 mg/mL by PBS and 0.85% NaCl aqueous solution at 37° C. for 24 h were also measured. The chitosan particles aggregated slightly in salt solutions. The size of chitosan was 33 nm in 0.85% NaCl aqueous solution and even larger in PBS solution (91 nm) (FIG. 4c). The zeta potentials of chitosan were 10.6 mV and 3.98 mV in 0.85% NaCl aqueous solution and PBS solution, respectively (FIG. 4d).

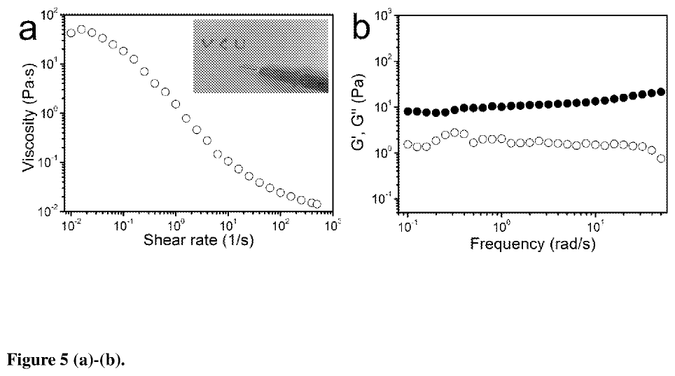

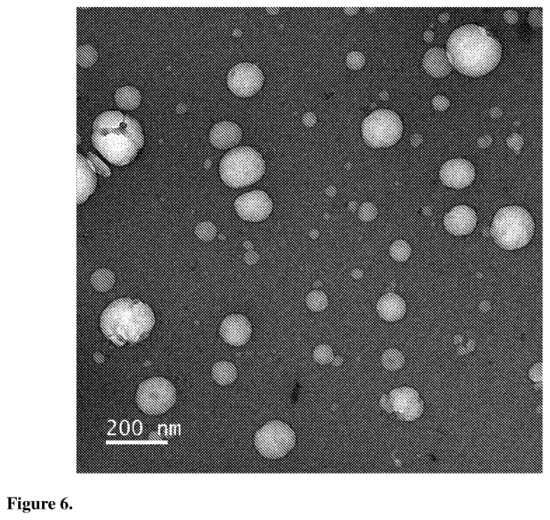

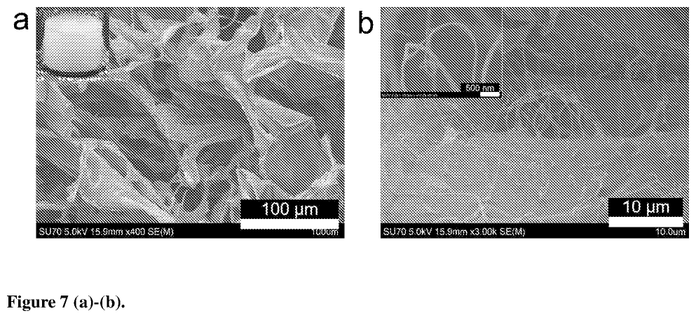

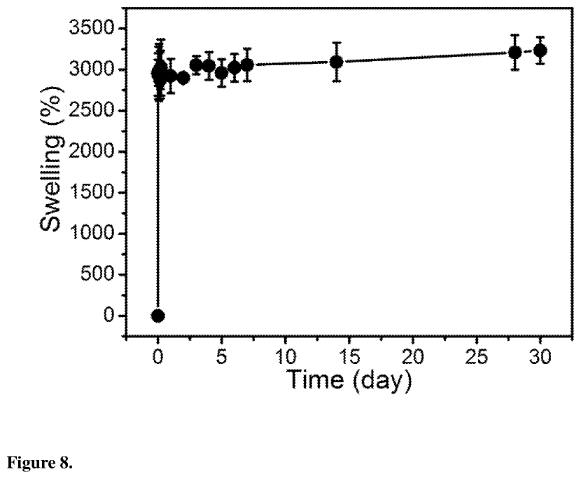

The chitosan pseudo-solution was shown to have a shear thinning property. The viscosity of the solution decreased by four orders of magnitude as the shear rate increased from 0.01 to 500 l/s (FIG. 5a). The water-based chitosan pseudo-solution was also injectable. It was pushed out of the syringe without clogging the needle (FIG. 5a inset), thus showing excellent processability. As shown in FIG. 5b, the oscillatory frequency sweep test elucidates that the solution has a slightly higher storage modulus (G′) than loss modulus (G″), and G′ is practically independent of frequency. The storage modulus is about 10 Pa. These features are typical of a weak hydrogel and are attributed to the significantly weakened hydrogen-bonding interactions among nano-sized chitosan. Chitosan nanoparticles (˜10-100 nm) were directly seen after the chitosan pseudo-solution was air-dried (FIG. 6). Upon freeze-drying, nano/submicron fibrous filaments were found within the chitosan foam (FIG. 7), complementarily reflecting the chitosan's nanostructures in solution. The larger size of chitosan nanostructures (particles/fibrous filaments) are due to the drying-induced aggregation. Nonetheless, the recovered chitosan became insoluble in the water presumably due to the restored hydrogen-bonding networks among the chitosan molecules. The material quickly reached the equilibrium swelling within 10 min and remained stable for at least 30 days (FIG. 8).

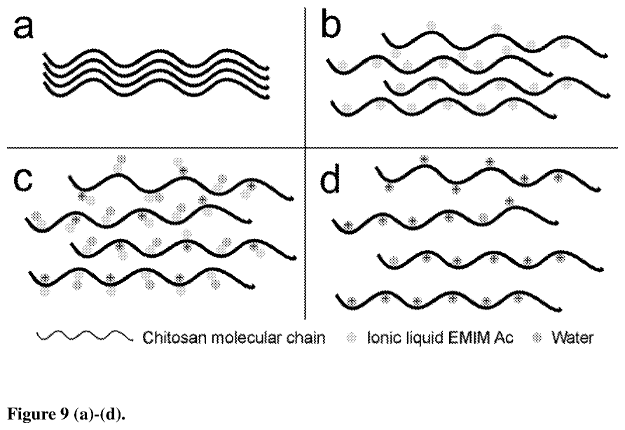

In this method, the hydrogen-bonded network of chitosan is first broken using the ionic liquid EMIM Ac under 120° C. The amines that participate in hydrogen bonding are freed consequently. The intermediate freezing/quenching step at −20° C. possibly freezes the chitosan solutes and prevents the dissolved chitosan from reconnecting with hydrogen-bonding interactions. During the solvent exchange process, the medium gradually changing to water (pH=7.22) leads to the protonation of chitosan's amine groups. The protonation of chitosan amino groups is believed to be vital in this process. It further reduces the likelihood of forming a hydrogen-bonded network by conferring electrostatic repulsion upon chitosan particles aggregation and promoting chitosan solvation (FIG. 9). Nonetheless, the protonation of amine groups is also susceptible to salt effects, which may disturb the stability of the obtained chitosan pseudo solutions as observed in our studies.

A pseudo-solution of nano-sized chitosan in plain water can be readily prepared by dissolving chitosan in the ionic liquid EMIM Ac followed by freezing at −20° C. and subsequent solvent exchange with water. The overall process is simple and eco-friendly. The obtained chitosan solution shows excellent colloidal stability and can be used directly as a weak liquid gel because of its shear thinning property. The results observed herein are unexpected and surprising, and provide for the successful dissolving and processing chitosan in plain water and further making chitosan-based products in biomedical and other applications.

The contents of each article, patent document, and publication mentioned herein are hereby incorporated by reference for all purposes.

1. A method for producing a composition comprising chitosan and water, comprising:
dissolving chitosan powder in an ionic liquid, to prepare a first composition comprising chitosan and ionic liquid; and
contacting the first composition with water under conditions sufficient to solvent-exchange all or substantially all of the ionic liquid with water;
to form a composition comprising chitosan and water.

2. The method of claim 1, further comprising, after the dissolving and prior to the contacting, freezing the first composition to form a frozen composition comprising chitosan and the ionic liquid, wherein said contacting the first composition comprises contacting the frozen composition.

3. The method of claim 1, further comprising vortexing the composition comprising chitosan and water.

4. The method of claim 1, wherein the composition comprising chitosan and water comprises chitosan solutes having a size in the range of 1 to 1,000 nm.

5. The method of claim 1, further comprising removing excess water from the composition comprising chitosan and water.

6. The method of claim 1, wherein the ionic liquid is hydrophilic.

7. The method of claim 1, wherein the ionic liquid is one or more of 1-allyl-3-methyl-imidazolium bromide; 1-allyl-3-methyl-imidazolium chloride; 1-butyl-3-methyl-imidazolium acetate; 1-butyl-3-methyl-imidazolium chloride; 1-ethyl-3-methyl-imidazolium acetate; 1-ethyl-3-methyl-imidazolium chloride; 1-ethyl-3-methyl-imidazolium dimethyl phosphate; 1-carboxymethyl-3-methylimidazolium chloride; hydrophilic ionic liquid; hydrophilic ionic liquid system; or any combination thereof.

8. The method of claim 1, wherein the ionic liquid is 1-ethyl-3-methyl-imidazolium acetate.

9. The method of claim 1, wherein a molecular weight (wt. avg.) of the chitosan ranges from about 5,000 to about 400,000 Da.

10. The method of claim 1, wherein a molecular weight (wt. avg.) of the chitosan is low molecular weight (50,000-190,000 Da), medium molecular weight (190,000-310,000 Da), or high molecular weight (310,000-375,000 Da).

11. A composition, comprising chitosan and water prepared by the process of claim 1.

12. The composition of claim 11, which consists essentially of chitosan and water.

13. The composition of claim 11, which consists of chitosan and water.

14. The composition of claim 11, which is in the form of solution, pseudo-solution, colloid, liquid, hydrogel, injectable gel, film, foam, nanofiber, porous scaffold, or particle.

15. The composition of claim 11, which is optically transparent.

16. The composition of claim 11, further comprising a benefit compound.

17. The composition of claim 11, further comprising a benefit compound selected from the group consisting of a pharmaceutically active ingredient, drug, hydrophobic drug, hydrophilic drug, biomolecule, medicine, cosmetic, food, or combination thereof.

18. A composition, comprising nano-sized chitosan solutes and water.

19. The composition of claim 18, wherein the chitosan solutes have a size in the range of 1 to 1,000 nm.

20. The composition of claim 18, wherein the chitosan solutes are stable and uniform.
